(19) United States
(12) Patent Application Publication
(10) Pub. No.: US 2006/0161257 A1
(43) Pub. Date: Jul. 20, 2006

(54) Pair of intervertebral prostheses

(75) Inventor: Arnold Keller, Kayhude (DE)

Correspondence Address: MORRISON & FOERSTER LLP, 1650 TYSONS BOULEVARD, SUITE 300, MCLEAN, VA, 22102, US

(73) Assignee: Cervitech, Inc., Rockaway, NJ (US)

(21) Appl. No.: 11/296,335

(22) Filed: Dec. 8, 2005

(30) Foreign Application Priority Data
Jan. 17, 2005 (EP) 05000841.6

(51) Int. Cl. A61F 2/44

(52) U.S. Cl. 623/17.11; 623/17.15

(57) ABSTRACT

A pair of intervertebral prostheses is configured to be implanted above and below a given vertebral body and each has an end plate configured for connection to this vertebral body and a securing flange which is assigned to the end plate and is configured for connection to the vertebral body from the ventral direction in an implanted position. To ensure that the securing flanges also have enough space in front of vertebral bodies of low height, they are offset relative to one another in such a way that one securing flange is arranged centrally, and the other securing flange is arranged eccentrically in a symmetrical configuration. The offset can be provided in the lateral direction and/or in the anterior-posterior direction relative to the implanted position.

### FIELD AND BACKGROUND OF THE INVENTION

It is sometimes necessary for two adjacent intervertebral disks in the spinal column to be replaced prosthetically. Two intervertebral prostheses are then placed above and one below the same vertebral body, each of them being connected to this vertebral body via one of their two end plates. If there is a risk of the end plates not having sufficient stability on the vertebral body, those prostheses are preferred which are secured on the vertebral body by means of ventral flanges. If the vertebral body has a low height, as is regularly the case in the cervical spine, the flanges belonging to the end plates to be secured on the top and bottom of the same vertebral body may collide with one another on the ventral face of the vertebral body. In order to avoid this, it is known (from US 2004/0249465 A; US 2004/0220590 A) to arrange the securing flanges offset from one another in the lateral direction. This has the disadvantage that the forces transmitted by the securing flange are introduced asymmetrically into the prosthesis, which may in some cases lead to a skewed position.

A substantially rigid intervertebral prosthesis is known (WO 01/62190) with a securing flange which projects downward at the center from the anterior face of the upper cover plate and which is to be connected to the adjacent vertebral body below it, and with two securing flanges which protrude upward from the anterior face of the lower cover plate which are to be connected to the upwardly adjacent vertebral body. It gives no indication as to how to configure the securing flanges of two different prostheses which engage on one and the same vertebral body.

### SUMMARY OF THE INVENTION

The invention avoids this disadvantage of the prior art by virtue of the features of this invention, in which one securing flange is arranged centrally, and the other is arranged eccentrically in a symmetrical configuration. In an advantageous embodiment of the invention, the securing flanges are offset relative to one another in the anterior-posterior (hereinafter “AP”) direction, with one securing flange advantageously overlapping the other. The overlapped securing flange is held securely by the other, so that only the latter has to be secured on the bone and provided with a screw hole.

Each securing flange can include at least one screw hole.

In the simplest embodiment, the securing flanges lie ventrally in front of the vertebral body concerned. Instead of this, it is also possible to fit the securing flanges into a ventral depression in the vertebral body. In this way it is possible to avoid irritation of the esophagus and of other organs lying in front of the cervical spine.

The securing flanges can be formed integrally with the associated end plates. However, they can also be separate parts.

### BRIEF DESCRIPTION OF THE DRAWINGS

The invention is explained in more detail below with reference to the drawing, in which:

FIG. 1 shows an arrangement according to the prior art, and

FIGS. 2 to 4 show embodiments according to the invention, namely

FIG. 2 a ventral view,

FIG. 3 a sectional view along the line V-V in FIG. 2,

FIG. 4 an alternative to the embodiment according to FIG. 3.

### DETAILED DESCRIPTION OF THE INVENTION

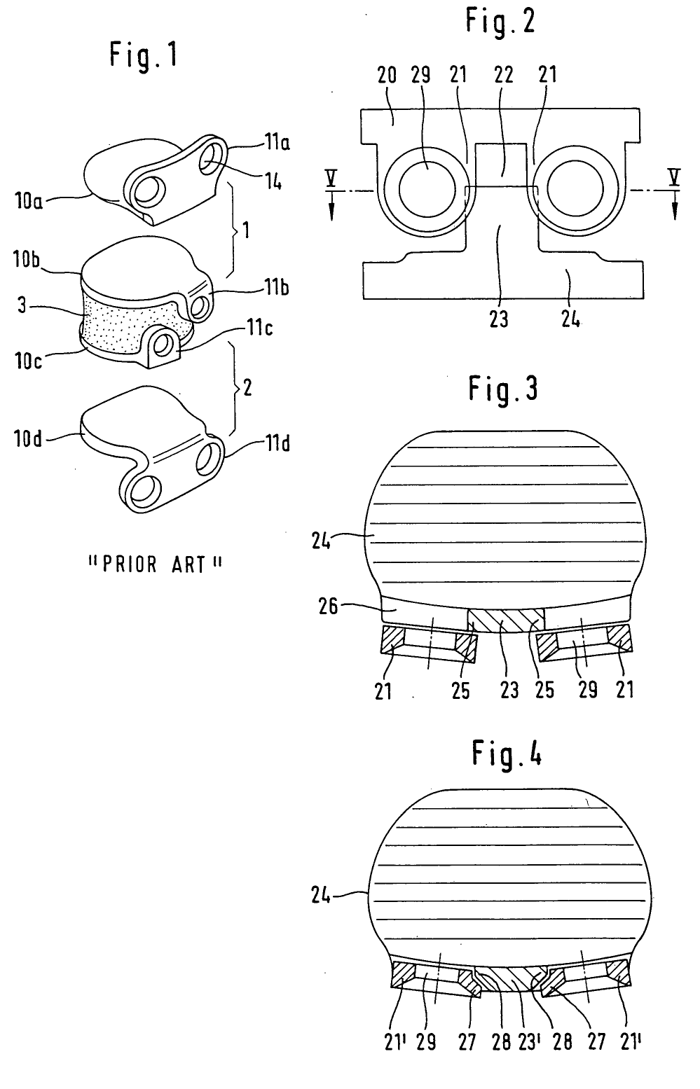

The intervertebral prostheses 1, 2 of the known arrangement according to FIG. 1, which are each shown separated, are each to be implanted in an intervertebral space above and below, respectively, the diagrammatically indicated vertebral body 3. They are made up of upper and lower end plates 10 which interact via articulating surfaces (not shown). At their front edges, they are connected permanently and in one piece with a securing flange 11. The upper end plate 10a of the upper prosthesis 1 has a centrally disposed, wide securing flange 1 a with two screw holes 14. The securing flange 11b of the lower end plate 10b is arranged eccentrically in order to provide room for the securing flange 11c of the upper end plate 10c of the lower prosthesis 2, which securing flange 11c is correspondingly offset toward the other side. The securing flange 11d of the lower end plate 10d of the lower prosthesis 2 is arranged centrally and wide. The asymmetrical configuration of the securing flanges 11b and 11c is disadvantageous. It is avoided by the symmetrical arrangement according to the invention, as is shown in FIGS. 2 to 4. The depiction shown is limited to those end plates of the two prostheses interacting with the vertebral body 3, and to their securing flanges. The end plates not shown can be similarly or otherwise configured.

On the upper end plate 20 shown in the drawing (this is the lower end plate of the upper prosthesis), two symmetrical securing flanges 21 are secured which enclose an interspace 22 in which is accommodated a centrally disposed securing flange 23 belonging to the lower end plate 24 in the drawing (this is the upper end plate of the lower prosthesis). If this central securing flange 23 is narrower than the interspace 22, the securing flanges 21, 23 are only laterally offset relative to one another. FIG. 2 shows an embodiment in which there is additionally an offset in the AP direction (AP=anteroposterior). The lateral edges 25 of the central securing flange 23 engage behind the corresponding edges of the securing flanges 21.

It is in this way fixed on the vertebral body by the securing flanges 21 lying in front of it. It therefore does not need to include any screw hole. It will be seen from FIG. 3 that the securing flanges 21, 23 take up a not inconsiderable amount of space in the AP direction. In order to minimize this space, it is possible to recess the central securing flange 23 in the front of the vertebral body into which, for this purpose, a corresponding depression has been worked prior to the insertion of the prosthesis. In other words, the securing flange 23 is integrated into the AP dimension of that part of the implant which is accommodated inside the intervertebral space. The same is done with the slightly raised edge, which is disposed at the ventral edge of the end plate 24, in order to form an obstacle against undesired dorsal displacement of the implant.

A variant of this embodiment is shown in FIG. 4, in which the edges 27, 28 of the securing flanges 21′, 24′ are undercut in such a way that they are accommodated in the same plane. The edges are formed in such a way that they each fit into the undercut of the other edge. In this form, the securing flanges can lie in front of the vertebral body or can be recessed into the latter. In the example shown, the two outer flanges 21′ are provided with screw holes 29, while the inner flange 23′ has no screw hole, since it is held by the outer flanges 21′ by virtue of the mutual form-fit engagement of the flange edges 27, 28. The arrangement could also be reversed, with the edges of the inner flange 23′ engaging over the edges of the outer flanges 21′ and the inner flange being provided with a screw hole 29. In the example shown, both securing flanges 21′ and 23′ are provided with an undercut. It may also be sufficient if only one of the two is provided with an undercut. For example, the securing flange 23 according to FIG. 3 can be combined with a securing flange 21′ according to FIG. 4.

In the preferred embodiment, the securing flanges 21, 23 are connected integrally to the associated end plates 21, 22. This does not have to be the case. Instead, they can be separate parts which engage with their free edge over the associated end plate 20, 24 and in this way secure the latter in the intervertebral space.

1. A pair of intervertebral prostheses which are configured to be implanted in a subject above and below a given vertebral body,
each prosthesis of the pair of intervertebral prostheses comprising an end plate configured to be connected to the vertebral body and a securing flange for the end plate that is configured to be connected to the vertebral body from a ventral direction relative to an implanted position in the subject of the pair of intervertebral prostheses,
the securing flanges being offset relative to one another in a lateral direction relative to the implanted position, one securing flange being arranged centrally and the other securing flange being arranged eccentrically in a symmetrical configuration.

2. The pair of intervertebral prostheses as claimed in claim 1, wherein each of the securing flanges includes at least one screw hole.

3. The pair of intervertebral prostheses as claimed in claim 1, wherein the securing flanges are offset relative to one another in an anterior-posterior direction relative to the implanted position.

4. The pair of intervertebral prostheses as claimed in claim 1, wherein one securing flange overlaps the other.

5. The pair of intervertebral prostheses as claimed in claim 4, wherein at least one of the securing flanges is undercut, in the area of the overlap, in order to receive part of the other securing flange.

6. The pair of intervertebral prostheses as claimed in claim 4, wherein the ventrally overlapping securing flange is provided with at least one screw hole.

7. The pair of intervertebral prostheses as claimed in claim 1, wherein at least one of the two securing flanges is configured for insertion into a depression formed in the vertebral body.

8. The pair of intervertebral prostheses as claimed claim 1, wherein the securing flanges are permanently connected to the respectively associated end plate.

9. The pair of intervertebral prostheses as claimed in claim 1, wherein the securing flanges are parts separate from the associated end plate.

10. The pair of intervertebral prostheses as claimed in claim 2, wherein one securing flange overlaps the other.

11. The pair of intervertebral prostheses as claimed in claim 10, wherein at least one of the securing flanges is undercut, in the area of the overlap, in order to receive part of the other securing flange.

12. The pair of intervertebral prostheses as claimed in claim 10, wherein only the ventrally overlapping securing flange is provided with at least one screw hole.

13. The pair of intervertebral prostheses as claimed in claim 2, wherein at least one of the two securing flanges is configured for insertion into a depression formed in the vertebral body.

14. The pair of intervertebral prostheses as claimed claim 2, wherein the securing flanges are permanently connected to the respectively associated end plate.

15. The pair of intervertebral prostheses as claimed in claim 2, wherein the securing flanges are parts separate from the associated end plate.

16. The pair of intervertebral prostheses as claimed in claim 2, wherein the securing flanges are offset relative to one another in an anterior-posterior direction relative to the implanted position.
